(19) United States
(12) Patent Application Publication
(10) Pub. No.: US 2024/0000393 A1
(43) Pub. Date: Jan. 4, 2024

(54) REALTIME ECG SIGNAL QUALITY ESTIMATION

(71) Applicant: Analog Devices International Unlimited Company, Limerick (LK)

(72) Inventors: Sudheer BABU, Therlam mandal (IN); Harsh BOLIA, Bangalore (IN); Gopal Gudhdur KARNAM, Koenigsbrunn (DE)

(21) Appl. No.: 18/037,953

(22) PCT Filed: Nov. 19, 2021
(86) PCT No.: PCT/EP2021/082312

§ 371 Date: May 19, 2023

(30) Foreign Application Priority Data
Nov. 19, 2020 (IN) 202041050373
Nov. 19, 2021 (WO) PCT/EP2021/082312

(51) Int. Cl. A61B 5/00; A61B 5/366; A61B 5/308

(52) U.S. Cl. CPC A61B 5/7221; A61B 5/366; A61B 5/6893; A61B 5/7203; A61B 5/308

(57) ABSTRACT

System and apparatus for real time electrocardiogram signal quality estimation. Systems and methods are disclosed for real time detection of corrupted segments of an electrocardiogram input signal and real time generation of an electrocardiogram based on an uncorrupted input signal. Systems and methods are provided for receiving a signal from an electrode, detecting corruption in a portion of the signal, and discarding the portion of the signal including the corruption.

### CROSS-REFERENCE TO RELATED APPLICATIONS

This application claims benefit of and priority to India Application No. 202041050373, titled “Realtime ECG Signal Quality Estimation”, which is hereby incorporated by reference in its entirety.

### FIELD OF THE DISCLOSURE

The present disclosure relates to electrocardiograms, and, more specifically, this disclosure describes apparatuses and systems for electrocardiogram signal quality estimation.

### BACKGROUND

Electrocardiograms are graphs showing electrical activity of the heart. In particular, electrical changes caused by depolarization and repolarization of the cardiac muscle are recorded using electrodes placed on the skin of a patient. Electrocardiograms show voltage over time. Single lead ECG recorders produce an ECG based on one lead, as compared to the 12 leads often used in medical office ECG recordings. In some examples, smart watches and other consumer health products include an ECG option that produces an ECG based on a single lead ECG recorder.

An electrocardiogram includes three main components: a P wave, a QRS complex, and a T wave. The P wave represents the depolarization of the atria. The QRS complex represents the depolarization of the ventricles, and includes a Q wave, an R wave, and an S wave. The Q wave is a downward deflection following the P wave, and the R wave follows the Q wave as an upward deflection. The S wave is a downward deflection following the R wave. The T wave is an upward deflection following the S wave and represents the repolarization of the ventricles.

This overview is intended to provide an overview of subject matter of the present patent application. It is not intended to provide an exclusive or exhaustive explanation of the invention. Further limitations and disadvantages of conventional and traditional approaches will become apparent to one of skill in the art, through comparison of such systems with some aspects of the present invention as set forth in the remainder of the present application with reference to the drawings.

### SUMMARY OF THE DISCLOSURE

System and apparatus are provided for detecting corrupted segments of an electrocardiogram in real time. In particular, systems and methods are provided for real time signal quality estimation for single lead electrocardiograms. In some examples, the single lead electrocardiograms are acquired from a steering wheel mounted electrode. Electrocardiograms acquired from single lead electrodes are riddled with motion artefacts and other noise. Systems and methods are provided herein to identify and reject segments corrupted by noise components in real time.

According to one aspect, a method for detecting corrupted segments of an electrocardiogram input signal in real time, comprises: receiving the input signal from a dry electrode; processing a first portion of the input signal, wherein processing includes identifying peaks; detecting corruption in at least a second portion of the input signal as the input signal is received; discarding the detected peaks in corrupted regions of the second portion of the input signal; and generating an electrocardiogram in real time with the good peaks identified.

In some implementations, receiving the input signal comprises receiving electrical activity from a single lead coupled to the dry electrode. In some implementations, the single lead is attached to a steering wheel. In some implementations, processing the first portion of the signal includes identifying peaks corresponding to the QRS complex type.

In some implementations, the method further comprises adaptively determining a threshold based on an amplitude of the peaks in the first portion of the signal. In some implementations, detecting corruption includes detecting noise exceeding the threshold. In some implementations, generating an ECG in real time including generating an ECG with a latency of one heartbeat. In some implementations, detecting corruption includes detecting low frequency noise. In some implementations, detecting corruption includes detecting narrowband powerline noise.

According to another aspect, a system for detecting corrupted segments of an electrocardiogram input signal in real time, comprises: a dry electrode configured to receive the input signal; a processor configured to: identify QRS peaks in a first portion of the input signal, detect corruption in at least a second portion of the input signal, discard the second portion of the input signal including the corruption, and generate an electrocardiogram in real time based on the identified QRS peaks.

In some implementations, the system further comprises an electrocardiogram lead coupled to the dry electrode and configured to receive electrical activity. In some implementations, the electrocardiogram lead is attached to a steering wheel. In some implementations, the processor is further configured to adaptively determine a threshold based on an amplitude of the peaks in the first portion of the signal and wherein corruption includes noise exceeding the threshold.

In some implementations, the processor generates the electrocardiogram with a latency of one heartbeat from the received input signal. In some implementations, the processor is configured to detect low frequency noise in the input signal and determine when the low frequency noise exceeds a threshold. In some implementations, the processor is configured to detect narrowband powerline noise in the input signal and determine when the narrowband powerline noise exceeds a threshold.

According to another aspect, a method for detecting corrupted segments of an electrocardiogram input signal in real time, comprises: receiving the input signal from an electrocardiogram lead coupled to a dry electrode; filtering the input signal into a low frequency component and a narrowband component; determining a low frequency component amplitude and a narrowband component amplitude; determining whether the low frequency component amplitude and the narrowband component amplitude fall below a threshold; and when the low frequency component amplitude and the narrowband component amplitude fall below the threshold: identifying QRS peaks in the input signal, and generating an electrocardiogram in real time based on the QRS peaks.

In some implementations, the method further comprises determining the threshold based on average peak amplitude over a plurality of QRS peaks. In some implementations, the method further comprises, when at least one of the low frequency component amplitude and the narrowband component amplitude exceeds a threshold, discarding the signal without generating the electrocardiogram. In some implementations, generating an ECG in real time including generating an ECG with a latency of one heartbeat.

According to another aspect, the present disclosure discloses an apparatus for detecting corrupted segments of an electrocardiogram in real time.

According to another aspect, a method for detecting corrupted segments of an electrocardiogram in real time includes receiving a signal from an electrode, detecting corruption in at least a first portion of the signal and discarding the first portion of the signal including the corruption.

The drawings show exemplary ECG recording configurations and plots. Variations of these configurations and plots, for example, changing the positions of, adding, or removing certain elements from the configurations, are not beyond the scope of the present invention. The illustrated ECG recorders, components, configurations, and complementary devices are intended to be complementary to the support found in the detailed description.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

For a fuller understanding of the nature and advantages of the present invention, reference is made to the following detailed description of preferred embodiments and in connection with the accompanying drawings, in which:

FIG. 1 is a flow chart showing a method for providing a real-time ECG, in accordance with some embodiments of the disclosure provided herein;

FIG. 2 shows two graphs of a recorded signal from dry electrodes, in accordance with some embodiments of the disclosure provided herein;

FIG. 3 shows two graphs of a recorded signal from dry electrodes, in accordance with some embodiments of the disclosure provided herein;

FIG. 4 shows three graphs of an input signal, in accordance with various embodiments of the disclosure;

FIG. 5 shows three graphs of an input signal, in accordance with various embodiments of the disclosure;

FIG. 6 shows a block diagram of an ECG system for use in a vehicle, according to various embodiments of the disclosure; and

FIG. 7 shows exemplary steering wheel mounted electrodes in accordance with some embodiments of the disclosure provided herein.

### DETAILED DESCRIPTION

The present disclosure relates to electrocardiograms, and, more specifically, this disclosure describes apparatuses and systems for real time electrocardiogram signal quality estimation. In particular, systems and methods are provided for real time signal quality estimation for single lead electrocardiograms.

Single lead electrocardiograms can be acquired from electrodes mounted on a steering wheel in a vehicle. However, electrocardiograms acquired from steering wheel mounted electrodes are riddled with motion artefacts. In some examples, motion artefacts are caused in part by the driver's hands being frequently in motion. One major type of artefact includes large baseline shifts due to temporary breaks in contact with the electrodes. Another major type of artefact includes smaller baseline shifts due to smaller palm movements with electrodes still in contact. Both these artefacts can result in wrong R-peak detection, which in turn affects the calculated values of derived parameters such as heart rate and heart rate variability. Another artefact that leads to wrong R-peak detection is powerline noise. Additionally, muscle noise and other types of narrowband noise sources can cause artefacts. While some conventional systems and methods can provide an estimation of signal quality after a long period of time, systems and methods are needed for real-time estimation of signal quality. Thus, systems and methods are provided herein to identify and reject segments corrupted by noise components in real time.

ECG signal acquisition hardware has a leads-off detection feature that can detect a break in contact with electrodes, but that alone is insufficient to address all the artefacts. In some examples, there may be contact with the electrode but nevertheless have a corrupted or noisy signal. Thus, there is a need to estimate the signal quality in software and use that to reject corrupt parts of the signal. In particular, there is a need to reject corrupt parts of the signal in real time. Systems and methods are provided for identification of baseline wander artefacts and powerline noise artefacts in ECG signals. In various implementations, wavelet decomposition of the signal is performed and the strength of selected decomposition levels is compared with a threshold derived from QRS height.

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

Two indicators or signal quality based on frequency include (1) pSQI and (2) basSQI. According to one example:

pSQI=(power in 5-15 Hz band)/(power in 5-40 Hz band)

Thus, pSQI is the ratio of QRS power to high frequency power. Intuitively, this ratio is expected to fall when the ECG signal is corrupted by powerline noise or other high frequency artefacts. According to another example:

basSQI=(power in 1-40 Hz band)/(power in 0-40 Hz band)

Thus, basSQI is the ratio of baseline power to ECG signal power. Intuitively, this ratio is expected to fall when the ECG signal is corrupted by baseline disturbances.

In some implementations, the indices are compared to fixed thresholds or are fed as input features to a learning-based classifier, to characterize the signal as good or bad quality. These indicators work well if the signal window used for calculating spectral power is large enough to accommodate several QRS complexes (e.g., 10 secs or more). Otherwise, the ratio will tend to follow the instantaneous characteristics of the ECG signal. Thus, these signal quality indices are not suitable for a real-time implementation.

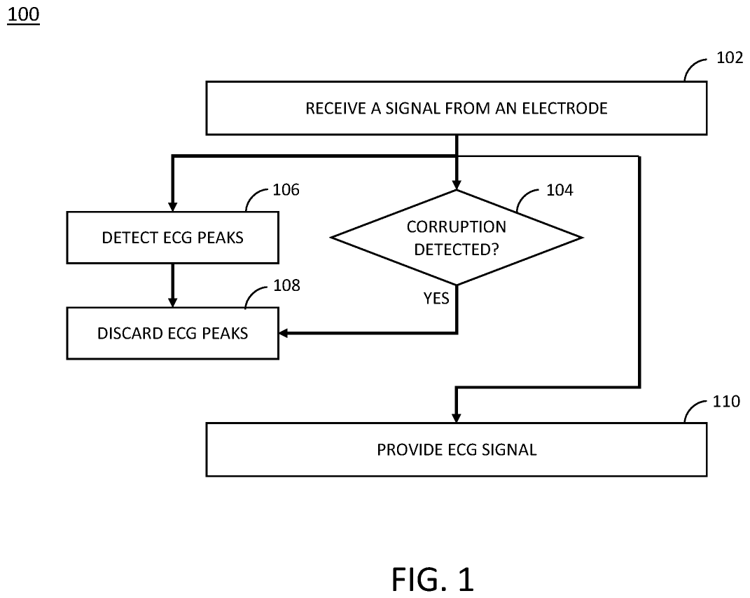

FIG. 1 is a flow chart illustrating a method 100 for providing a real-time ECG, according to various embodiments of the disclosure. At step 102, a signal is received from one or more dry electrodes. The dry electrodes are coupled to an ECG lead, and the signal received from the lead can include ECG signals as well as noise or other corruption. At step 104, it is determined whether signal corruption is detected in a first portion of the received signal. In some examples, the first portion is about a one second long portion, and in other examples, the first portion is less than about a one second long portion. In various examples, the first portion is long enough to include one QRS pulse.

At step 104, the method 100 identifies signal corruption that is strong enough to prevent accurate QRS complex peak detection, as described in greater detail below. If, at step 104, corruption is detected in the first portion of the signal, the method 100 proceeds to step 108 and any identified peaks in the corrupted portion of the signal are discarded. If no corruption is detected at step 104, the method 100 proceeds, and at step 110, a real-time ECG signal is provided based on the received signal. Note that as long as a signal is continuously received at step 102 from an ECG lead, the method 100 continues to provide a real-time ECG at step 110 based on the received signal.

When a corrupted signal is detected at step 104, the peak detector output can be temporarily suspended until a non-corrupted signal is received. In particular, at step 106, ECG peaks in the received signal are detected and identified. However, if at step 104, signal corruption is detected in a first portion of the signal, the detected peaks in the first portion of the signal are discarded at step 108.

In various implementations, similarity in the shape of QRS complexes can be used to further identify signal quality. This is used as an additional check to avoid false detection of noise peaks that resemble QRS complex in their shape. In particular, the shape of QRS complex corresponding to a normal heart beat, for an individual, should be consistent. In some examples, a template is constructed with four consecutive QRS complexes that are similar to each other; QRS complexes that are not similar to the template are discarded. If more than four consecutive QRS complexes do not match with the template, the template is updated.

Estimation of Strength in the Frequency Band of Corrupting Noise

In various implementations, in order to extract the strength in the frequency region of each corrupting noise, discrete wavelet transform is used. In some examples, other digital filtering methods are used. The amplitude of the signal is filtered into the following bands:
(1) A low frequency component: less than 2 Hz for baseline estimation. In some examples, the low frequency noise component is less than about 5 Hz.
(2) A narrowband component: matching the frequency of the disturbance. In some examples, a 50 Hz band is selected. A 50 Hz band corresponds to powerline noise. In other examples, the narrowband component includes one or more frequency bands between 50 Hz-60 Hz.

Estimating the Baseline Deviation (low frequency noise)

In various implementations, the low pass filtered signal by itself cannot be used as an instantaneous baseline estimate because the filtered signal has a larger amplitude at QRS locations. Thus, median filtering is applied to flatten out the signal in a short window (e.g., 1.5 seconds). Then the maximum deviation within the window is used as an estimate of the low frequency disturbance.

Estimating Narrowband Noise Components

In various implementations, the mean of the absolute value of signal amplitude in the relevant frequency band (50-60 Hz for powerline noise), calculated in a short window, is used as an estimate of the narrowband disturbance. This way the instantaneous oscillations are ignored.

Computation of an Adaptive Threshold to use as Reference

According to various implementations, a fixed threshold is not used since the components in each band can vary with heart rate and QRS amplitude. Additionally, ECG values vary from person to person. Thus, the average QRS amplitude itself is used as a threshold. In one example, the average of last five good beats is used as the threshold. QRS amplitude is taken as the difference between maximum and minimum values in a small window centered around the r-peak location. In some examples, the adaptive threshold can be used to determine whether the signal is corrupt.

Real-time Signal Quality Estimation

According to various implementations, only a small latency is introduced in estimating the signal quality. In some examples, a latency of about 1.5 seconds is used, where 1.5. seconds is approximately the duration of two heart beats. Thus, signal quality is estimated in real time. In some examples, the latency is about 0.75 seconds, where 0.75 seconds is approximately the duration of one heartbeat. In various examples, the latency is one or two heartbeats, and in some examples, the latency varies based on heart rate.

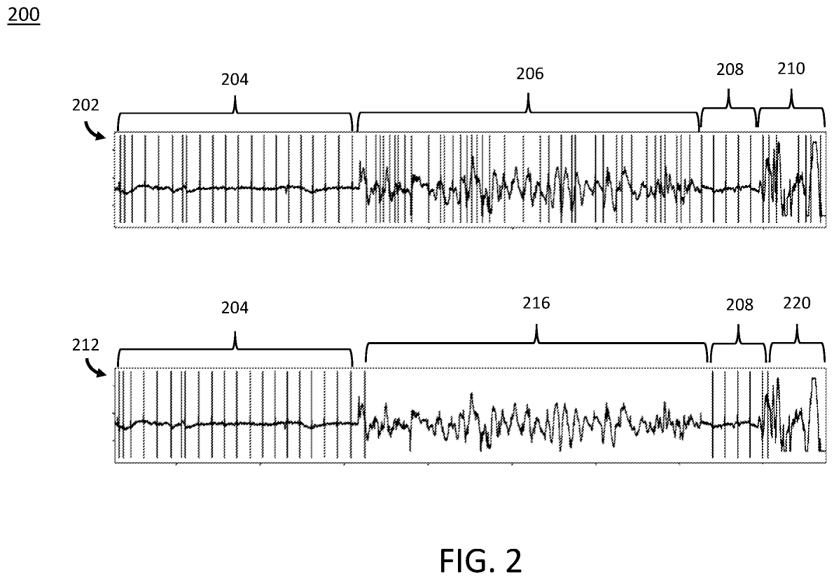

FIG. 2 shows two graphs of an input signal from ECG electrodes, in accordance with some embodiments of the disclosure provided herein. The top graph 202 shows an example of wrong peak detection due to low frequency noise components. In particular, each vertical line is a peak detection line. In the first section 204, the signal is not noisy and the peaks are accurately detected. In the second section 206, the signal is noisy obscuring QRS peaks. However, the system attempts to detect peaks despite the noise, resulting falsely-identified peaks and an inaccurate ECG output. The third section 208 is again a clean signal without significant noise, in which the QRS peaks are easily detected. In the fourth section 210, the signal is again obscured by noise and peaks are not accurately identified.

The lower graph 212 in FIG. 2 shows an example in which the systems and methods disclosed herein are applied to the ECG signal. Thus, during the first section 204, the input signal is not noisy and the peaks are accurately detected. At the end of the first section, the system identifies noise in the signal, and during the second section 216, the system discards peak detector output. At the end of the second section 216, the system identifies that the signal is no longer corrupted by noise. In the third section 208, the system again identifies QRS peaks in the signal and generates an ECG. At the end of the third section 208, the system again identifies noise in the signal, and during the fourth section 220, the system does not attempt to identify peaks and does not output updated ECG information. Thus, in this manner, the system provides a real-time ECG so long as it receives an uncorrupted signal.

In contrast, other systems do not provide real-time ECGs. In some systems, signals are collected and sent to a remote computing system and/or cloud for processing, generating a delayed ECG. In some systems, signals are collected over a period of time and later processed to generate an ECG.

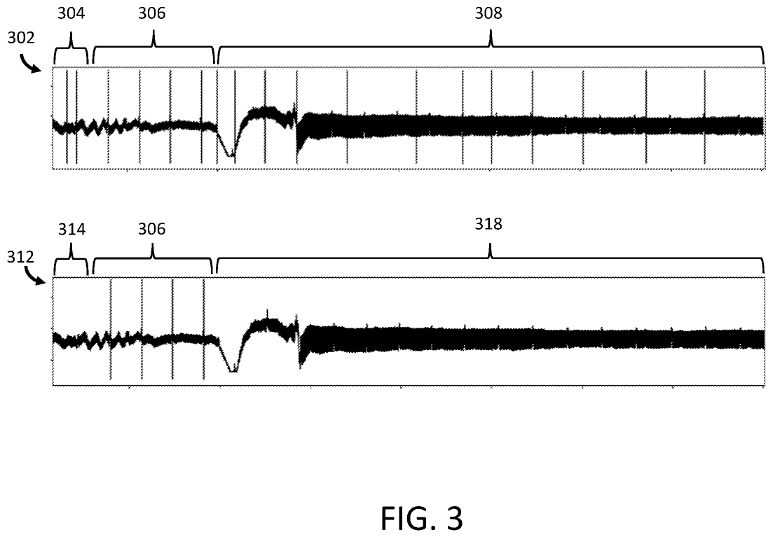

FIG. 3 shows two graphs of a recorded signal from dry electrodes, in accordance with some embodiments of the disclosure provided herein. The top graph 302 shows an example of wrong peak detection due to high frequency noise components. In particular, each vertical line is a QRS peak detection line. An initial noisy section 304 includes many falsely-identified peaks, as does a second noisy section 308. In between, there is a short section 306 in which the signal is uncorrupted and peaks are correctly identified.

The lower graph 312 in FIG. 3 shows an example in which the systems and methods disclosed herein are applied to the ECG signal. During the initial section 314, corruption is detected in the signal and any identified peaks are rejected. During the middle section 306, the input signal is not noisy and the peaks are accurately detected to generate an ECG. At the end of the second section 306, the system identifies noise in the signal, and during the third section 316, the system does not accurately identify peaks and does not output updated ECG information. Thus, the lower graph 312 shows an example in which the noisy section with the high frequency noise components is identified and any identified peaks in the noisy section of the recording are rejected.

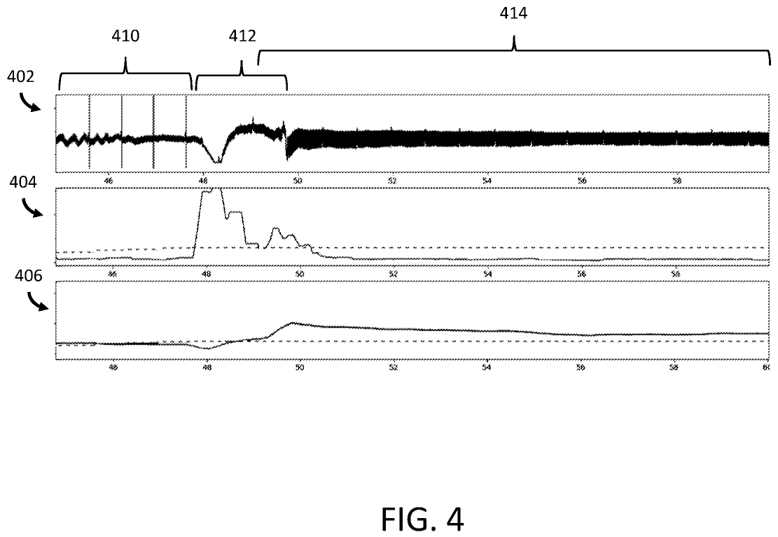

FIG. 4 shows three graphs, in accordance with various embodiments of the disclosure. The top graph 402 shows a noisy ECG signal. In particular, the first time window 410 of the ECG signal in the top graph 402 is not noisy, and QRS peaks are detected during this portion. However, during the following time periods including the second 412 and third 414 time windows, the ECG signal is noisy and no QRS peaks are recorded.

The middle graph 404 shows an estimation of the low frequency noise components (Q1), where the dashed line is the adaptive threshold derived from the QRS amplitude (T). As shown in the middle graph 404, the low frequency noise components exceed the adaptive threshold indicated by the dashed line during the second window of time 412. Thus, during the second window of time 412, QRS peaks are not recorded. While the low frequency noise components subsequently drop below the threshold indicated by the dashed line, the ECG signal includes other noise components that prevent accurate QRS peak detection, as shown with respect to the bottom graph 406.

The bottom 406 graph shows an estimation of narrowband noise components (Q2), where the dashed line is the adaptive threshold derived from the QRS amplitude (T). As shown in the bottom graph 406, the narrowband noise components exceed the adaptive threshold indicated by the dashed line during the third window of time 414. Note that in the example shown in FIG. 4, the third window of time 414 overlaps with the second window of time 412, and during the overlapping period, both the low frequency noise components and the narrowband noise components exceed their respective thresholds. Since, during the third window of time 414, the narrowband noise components exceed the threshold as shown in the bottom graph 406, ECG peaks are not generated for an ECG output during the third window of time 414, as shown in the top graph 402.

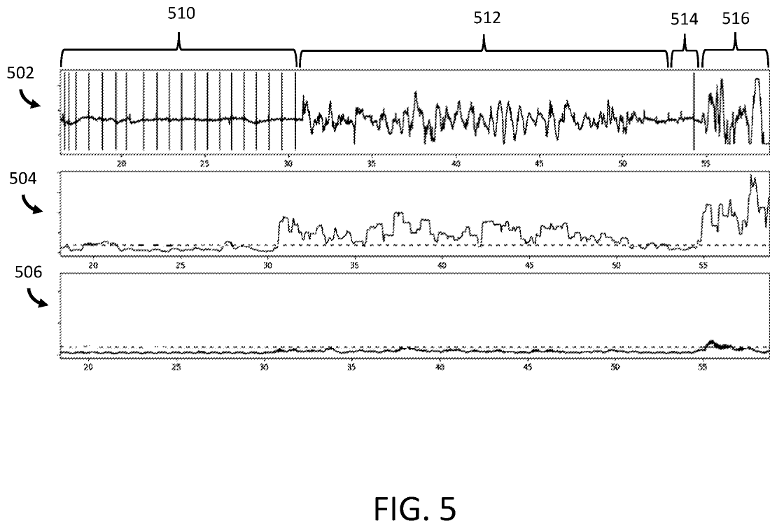

FIG. 5 shows three graphs, in accordance with various embodiments of the disclosure. The top graph 502 shows a noisy ECG signal. In particular, the first time window 510 of the ECG signal in the top graph 502 is not noisy, and QRS peaks are detected during this portion. However, during the following time period 512, the ECG signal is noisy and no QRS peaks are recorded. There is a short period of time during the third time window 514 during which the ECG signal is not noisy and a QRS peak is identified. Then, during the fourth time period 516, the ECG signal becomes noisy again, and no QRS peaks are recorded.

The middle graph 504 shows an estimation of a low frequency noise component (Q1), where the dashed line is the adaptive threshold derived from the QRS amplitude (T). As shown in the middle graph 504, the low frequency noise components exceed the adaptive threshold indicated by the dashed line during the second 512 and fourth 514 windows of time 512. Thus, during the second 512 and fourth 514 windows of time, QRS peaks are not recorded.

The bottom graph 506 shows an estimation of a narrowband noise component (Q2), where the dashed line is the adaptive threshold derived from the QRS amplitude (T). As shown in the bottom graph 506, the narrowband noise components exceed the adaptive threshold indicated by the dashed line for a small portion of the fourth time window 516. During the rest of the time, the narrowband noise components remain low, below the threshold, and do not prevent detection and identification of QRS peaks.

In general, systems and methods are provided herein for estimation of the strength of low frequency and narrow band noise components in ECG signal by digital filtering in the relevant band. Additionally, systems and methods are provided for removal of QRS features from the low pass filtered signal through median filtering. Rapid variations are smoothed out using averaging of the absolute value of narrowband components. Noise amplitude is compared with an adaptive threshold derived from QRS amplitude. Additionally, systems and methods are provided for real-time estimation of signal quality.

According to some implementations, an ECG is sampled at 500 Hz and decomposed using the a Daubechies D8 wavelet (also known as db4) up to level 7. A Daubechies D8 wavelet has eight coefficients. There are various approximation and detail coefficients at different levels of wavelet decomposition. In some examples, the approximation, or scaling, coefficients are the lowpass representation of the signal and the details are the wavelet coefficients. At subsequent levels, the approximation coefficients are divided into a coarse approximation (lowpass) part and a detailed (highpass) part. In some examples, an approximation at level J plus details at level J can be used to determine an approximation at level J−1. Approximation coefficients at level “n” are can be referred to as cAn or an, and detail coefficients at level “n” are can be referred to as cDn or dn.

In some implementations, a baseline estimate is formed from a7 and smoothed using median smoothing to remove a small leak of QRS features. In some implementations, a baseline noise (Q1) is determined using a difference between minimum and maximum value of smoothed baseline estimates in a window. In some implementations, a power line noise estimate is determined from d4 and d3. In some implementations, line noise (Q2) is determined using an average value of an absolute value of a powerline noise estimate in a window. In some implementations, QRS amplitude (T) is calculated around the R-peak position, identified using popular peak detection algorithm. In some implementations, Q1 and Q2 are compared to a threshold value alpha*T, with different alpha used for Q1 and Q2. According to some examples, ECG signal is considered to have good quality if Q1 and Q2 are less than their respective thresholds. Additionally, in some implementations, peaks from segments identified as noise free are used to update the QRS amplitude.

According to various implementations, wavelet transform is just one way of extracting signal amplitude/energy in a desired frequency band. Other methods of filtering can also be applied to achieve the outcome.

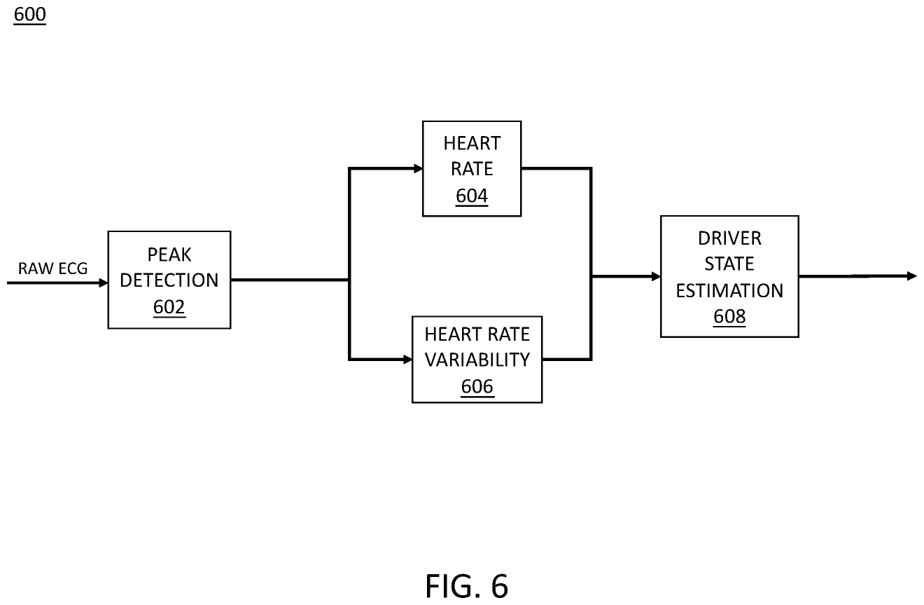

FIG. 6 shows a block diagram 600 of an ECG system for use in a vehicle, according to various embodiments of the disclosure. In some examples, the ECG system is used in a vehicle to evaluate driver state, and identify driver stress, driver drowsiness, and other conditions that can affect driving behavior and/or safety. A raw ECG signal is input to a QRS peak detection module 602. In some examples, the QRS peak detection module can be a computer system that runs a peak detection algorithm. The peak detection module 602 detects QRS peaks in real-time and functions as described above with respect to FIGS. 1-5. In various examples, the peak detection module 602 outputs QRS peak information and QRS peak locations. The output from the peak detection module 602 is used to determine user heart rate at the heart rate module 604, and well as heart rate variability at the heart rate variability module 606. The heart rate and heart rate variability can be used at the driver state estimation module 608 to evaluate driver state. In some examples, the driver state estimation module 608 is configured to identify driver drowsiness and/or driver stress. In some examples, the driver state estimation module 608 is configured to identify other driver states. In various implementations, the output from the driver state estimation module 608 is input to a vehicle system configured to attempt to prevent dangerous driving conditions. For example, the vehicle can be configured to alert a drowsy driver to wake up or pull over.

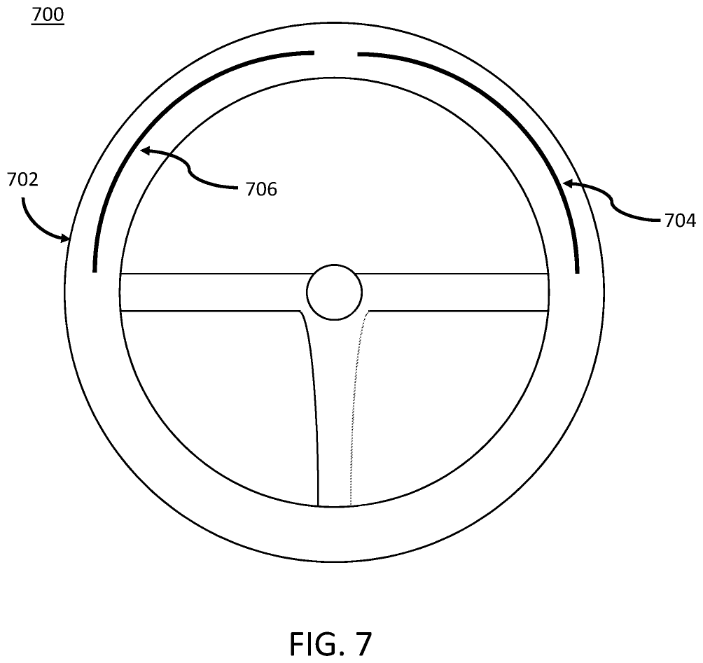

FIG. 7 shows exemplary steering wheel mounted dry electrodes in accordance with some embodiments of the disclosure provided herein. In particular FIG. 7 shows a steering wheel 702 having two mounted electrodes 704, 706. The electrodes 704, 706 are the ECG electrodes. In some examples, there is a third electrode, often referred to as a right leg drive electrode. The right leg drive electrode functions to improve signal to noise ratio (SNR) by reducing the common mode. The common mode is common in both the left and right electrodes, and in some examples, the common mode is powerline noise.

In some examples, the steering wheel electrodes 704, 706 are configured to determine if there is contact with one or both electrodes 704, 706 (e.g., if there is a hand in contact with the steering wheel). In some implementations, another set of two mounted electrodes 704, 706 is positioned elsewhere on the steering wheel 702. When a driver grasps the steering wheel 702 with both hands (one on each side of the top half of the wheel 702), one hand connects with a first electrode 704, and the other hand connects with the second electrode 706. In various implementations, an ECG can be recorded from just one set of electrodes. In particular, the two electrodes 704, 706 collect a single lead ECG. In some examples, there can be a different potential on each side (right hand side vs left hand side), and a lead can be used to ground the body at a known potential, as well as to measure a delta between the right and left hand side.

In various implementations, the dry electrodes can be a part of other types of systems. For example, the dry electrodes can be integrated into a smart watch and/or wrist band. In some examples, the dry electrodes can be integrated into a chest strap. In some examples, the dry electrodes can be integrated into handles on exercise equipment.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein.

Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

Note that the activities discussed above with reference to the FIGURES which are applicable to any integrated circuit that involves signal processing (for example, gesture signal processing, video signal processing, audio signal processing, analog-to-digital conversion, digital-to-analog conversion), particularly those that can execute specialized software programs or algorithms, some of which may be associated with processing digitized real-time data.

In some cases, the teachings of the present disclosure may be encoded into one or more tangible, non-transitory computer-readable mediums having stored thereon executable instructions that, when executed, instruct a programmable device (such as a processor or DSP) to perform the methods or functions disclosed herein. In cases where the teachings herein are embodied at least partly in a hardware device (such as an ASIC, IP block, or SoC), a non-transitory medium could include a hardware device hardware-programmed with logic to perform the methods or functions disclosed herein. The teachings could also be practiced in the form of Register Transfer Level (RTL) or other hardware description language such as VHDL or Verilog, which can be used to program a fabrication process to produce the hardware elements disclosed.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interfaces, or objects that facilitate the operations thereof.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof.

In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs. Further, the information being tracked, sent, received, or stored in a processor could be provided in any database, register, table, cache, queue, control list, or storage structure, based on particular needs and implementations, all of which could be referenced in any suitable timeframe.

Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a personal digital assistant (PDA), a smart phone, a mobile phone, an iPad, or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Computer-executable instructions may be in many forms, such as program modules, executed by one or more computers or other devices. Generally, program modules include routines, programs, objects, components, data structures, etc. that performs particular tasks or implement particular abstract data types. Typically, the functionality of the program modules may be combined or distributed as desired in various embodiments.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Computer program logic implementing all or part of the functionality described herein is embodied in various forms, including, but in no way limited to, a source code form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

In some embodiments, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc.

Other components such as external storage, additional sensors, controllers for audio/video display, and peripheral devices may be attached to the board as plug-in cards, via cables, or integrated into the board itself. In another example embodiment, the electrical circuits of the FIGURES may be implemented as standalone modules (e.g., a device with associated components and circuitry configured to perform a specific application or function) or implemented as plug-in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this disclosure.

In certain cases, it may be easier to describe one or more of the functionalities of a given set of flows by only referencing a limited number of electrical elements. It should be appreciated that the electrical circuits of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Select Examples

Example 1 provides a method for detecting corrupted segments of an electrocardiogram input signal in real time, comprising: receiving the input signal from a dry electrode; processing a first portion of the input signal, wherein processing includes identifying peaks; detecting corruption in at least a second portion of the input signal as the input signal is received; discarding the second portion of the input signal including the corruption; and generating an electrocardiogram in real time based on the peaks.

Example 2 provides a method according to any of the preceding and/or following examples, wherein receiving the input signal comprises receiving electrical activity from a single lead coupled to the dry electrode.

Example 3 provides a method according to any of the preceding and/or following examples, wherein the single lead is attached to a steering wheel.

Example 4 provides a method according to any of the preceding and/or following examples, wherein processing the first portion of the signal includes identifying peaks corresponding to the QRS complex type.

Example 5 provides a method according to any of the preceding and/or following examples, further comprising adaptively determining a threshold based on an amplitude of the peaks in the first portion of the signal.

Example 6 provides a method according to any of the preceding and/or following examples, wherein detecting corruption includes detecting noise exceeding the threshold.

Example 7 provides a method according to any of the preceding and/or following examples, wherein generating an ECG in real time including generating an ECG with a latency of one heartbeat.

Example 8 provides a method according to any of the preceding and/or following examples, wherein detecting corruption includes detecting low frequency noise.

Example 9 provides a method according to any of the preceding and/or following examples, wherein detecting corruption includes detecting narrowband powerline noise.

Example 10 provides a system for detecting corrupted segments of an electrocardiogram input signal in real time, comprising: a dry electrode configured to receive the input signal; a processor configured to: identify QRS peaks in a first portion of the input signal, detect corruption in at least a second portion of the input signal, discard the second portion of the input signal including the corruption, and generate an electrocardiogram in real time based on the identified QRS peaks.

Example 11 provides a system according to any of the preceding and/or following examples, further comprising an electrocardiogram lead coupled to the dry electrode and configured to receive electrical activity.

Example 12 provides a system according to any of the preceding and/or following examples, wherein the electrocardiogram lead is attached to a steering wheel.

Example 13 provides a system according to any of the preceding and/or following examples, wherein the processor is further configured to adaptively determine a threshold based on an amplitude of the peaks in the first portion of the signal and wherein corruption includes noise exceeding the threshold.

Example 14 provides a system according to any of the preceding and/or following examples, wherein the processor generates the electrocardiogram with a latency of one heartbeat from the received input signal.

Example 15 provides a system according to any of the preceding and/or following examples, wherein the processor is configured to detect low frequency noise in the input signal and determine when the low frequency noise exceeds a threshold.

Example 16 provides a system according to any of the preceding and/or following examples, wherein the processor is configured to detect narrowband powerline noise in the input signal and determine when the narrowband powerline noise exceeds a threshold.

Example 17 provides a method for detecting corrupted segments of an electrocardiogram input signal in real time, comprising: receiving the input signal from an electrocardiogram lead coupled to a dry electrode; filtering the input signal into a low frequency component and a narrowband component; determining a low frequency component amplitude and a narrowband component amplitude; determining whether the low frequency component amplitude and the narrowband component amplitude fall below a threshold; and when the low frequency component amplitude and the narrowband component amplitude fall below the threshold: identifying QRS peaks in the input signal, and generating an electrocardiogram in real time based on the QRS peaks.

Example 18 provides a method according to any of the preceding and/or following examples, further comprising determining the threshold based on average peak amplitude over a plurality of QRS peaks.

Example 19 provides a method according to any of the preceding and/or following examples, further comprising, when at least one of the low frequency component amplitude and the narrowband component amplitude exceeds a threshold, discarding the signal without generating the electrocardiogram.

Example 20 provides a method according to any of the preceding and/or following examples, wherein generating an ECG in real time including generating an ECG with a latency of one heartbeat.

Interpretation of Terms

All definitions, as defined and used herein, should be understood to control over dictionary definitions, definitions in documents incorporated by reference, and/or ordinary meanings of the defined terms. Unless the context clearly requires otherwise, throughout the description and the claims:

“comprise,” “comprising,” and the like are to be construed in an inclusive sense, as opposed to an exclusive or exhaustive sense; that is to say, in the sense of “including, but not limited to”.

“connected,” “coupled,” or any variant thereof, means any connection or coupling, either direct or indirect, between two or more elements; the coupling or connection between the elements can be physical, logical, or a combination thereof.

“herein,” “above,” “below,” and words of similar import, when used to describe this specification shall refer to this specification as a whole and not to any particular portions of this specification.

“or,” in reference to a list of two or more items, covers all of the following interpretations of the word: any of the items in the list, all of the items in the list, and any combination of the items in the list.

the singular forms “a”, “an” and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present) depend on the specific orientation of the apparatus described and illustrated. The subject matter described herein may assume various alternative orientations. Accordingly, these directional terms are not strictly defined and should not be interpreted narrowly.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Elements other than those specifically identified by the “and/or” clause may optionally be present, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “between” is to be inclusive unless indicated otherwise. For example, “between A and B” includes A and B unless indicated otherwise.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke 35 U.S.C. § 112(f) as it exists on the date of the filing hereof unless the words “means for” or “steps for” are specifically used in the particular claims; and (b) does not intend, by any statement in the disclosure, to limit this disclosure in any way that is not otherwise reflected in the appended claims.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

1. A method, the method comprising:
receiving an electrocardiogram input signal from a dry electrode;
processing a first portion of the electrocardiogram input signal, wherein processing includes identifying peaks;
detecting corruption in at least a second portion of the electrocardiogram input signal as the electrocardiogram input signal is received;
discarding the second portion of the electrocardiogram input signal including the corruption; and
generating an electrocardiogram in real time based on the peaks.

2. The method of claim 1, wherein receiving the electrocardiogram input signal comprises receiving electrical activity from a single lead coupled to the dry electrode.

3. The method of claim 2, wherein the single lead is attached to a steering wheel.

4. The method of claim 1, further comprising classifying a QRS complex type from the electrocardiogram input signal, and wherein processing the first portion of the electrocardiogram input signal includes identifying peaks corresponding to the QRS complex type.

5. The method of claim 1, further comprising adaptively determining a threshold based on an amplitude of the peaks in the first portion of the electrocardiogram input signal.

6. The method of claim 5, wherein detecting corruption includes detecting noise exceeding the threshold.

7. The method of claim 1, wherein generating an ECG in real time includes generating an ECG with a latency of one heartbeat.

8. The method of claim 1, wherein detecting corruption includes detecting low frequency noise.

9. The method of claim 1, wherein detecting corruption includes detecting narrowband powerline noise.

10. A system, comprising:
a dry electrode configured to receive an electrocardiogram input signal; and
a processor configured to:
identify QRS peaks in a first portion of the electrocardiogram input signal,
detect corruption in at least a second portion of the electrocardiogram input signal,
discard the second portion of the electrocardiogram input signal including the corruption, and
generate an electrocardiogram in real time based on the identified QRS peaks.

11. The system of claim 10, further comprising an electrocardiogram lead coupled to the dry electrode and configured to receive electrical activity.

12. The system of claim 11, wherein the electrocardiogram lead is attached to a steering wheel.

13. The system of claim 10, wherein the processor is further configured to adaptively determine a threshold based on an amplitude of the peaks in the first portion of the electrocardiogram input signal and wherein corruption includes noise exceeding the threshold.

14. The system of claim 10, wherein the processor generates the electrocardiogram with a latency of one heartbeat from the received electrocardiogram input signal.

15. The system of claim 10, wherein the processor is configured to detect low frequency noise in the electrocardiogram input signal and determine when the low frequency noise exceeds a threshold.

16. The system of claim 10, wherein the processor is configured to detect narrowband powerline noise in the electrocardiogram input signal and determine when the narrowband powerline noise exceeds a threshold.

17. A method, comprising:
receiving an electrocardiogram input signal from an electrocardiogram lead coupled to a dry electrode;
filtering the electrocardiogram input signal into a low frequency component and a narrowband component;
determining a low frequency component amplitude and a narrowband component amplitude;
determining whether the low frequency component amplitude and the narrowband component amplitude fall below a threshold; and
when the low frequency component amplitude and the narrowband component amplitude fall below the threshold:
identifying QRS peaks in the electrocardiogram input signal, and
generating an electrocardiogram in real time based on the QRS peaks.

18. The method of claim 17, further comprising determining the threshold based on average peak amplitude over a plurality of QRS peaks.

19. The method of claim 17, further comprising, when at least one of the low frequency component amplitude and the narrowband component amplitude exceeds the threshold, discarding the electrocardiogram input signal without generating the electrocardiogram.

20. The method of claim 17, wherein generating the electrocardiogram (ECG) in real time includes generating an ECG with a latency of one heartbeat.
